(19) United States
(12) Patent Application Publication
(10) Pub. No.: US 2006/0015066 A1
(43) Pub. Date: Jan. 19, 2006

(54) Intraosseous infusion device

(76) Inventors: Melanie J. Turieo, Arlington, MA (US); John F. Stokes, Lawrence, MA (US); David Nedder, Attleboro, MA (US); Robert P. Maloney, Lee, MA (US); Robert W. Etheredge III, Natick, MA (US); Theodore J. Wright, Malden, MA (US)

Correspondence Address: LOWRIE, LANDO & ANASTASI, RIVERFRONT OFFICE, ONE MAIN STREET, ELEVENTH FLOOR, CAMBRIDGE, MA, 02142, US

(21) Appl. No.: 11/131,340

(22) Filed: May 17, 2005

Related U.S. Application Data
(60) Provisional application No. 60/571,817, filed on May 17, 2004.

(51) Int. Cl. A61M 5/20

(52) U.S. Cl. 604/136

(57) ABSTRACT

A device for infusion or aspiration that includes a base and at least one needle positioned within the base, where the base includes one or more locators for positioning the infusion device in relation to one or more predetermined anatomical features. A device for infusion or aspiration that comprises a drive assembly and a plurality of needles positioned within the drive assembly. A method for performing infusion or aspiration that includes the steps of establishing the location of the surface of a bone using the tip of a needle and then driving the needle into the bone a predetermined distance from the established location of the surface of the bone.

### RELATED APPLICATIONS

This application claims priority under 35 U.S.C. § 119(e) to U.S. Provisional Application Ser. No. 60/571,817, entitled “Reusable Intraosseous Infusion Device,” filed on May 17, 2004, which is herein incorporated by reference in its entirety.

### FEDERALLY SPONSORED RESEARCH

Portions of this invention were made with government support under Small Business Innovation Research (“SBIR”) award number W81XWH-3314-M043. The Government may have certain rights to this invention.

### BACKGROUND OF INVENTION

1. Field of Invention

The present invention relates generally to medical devices and methods and, in particular, to devices and methods for intraosseous infusion or aspiration.

2. Discussion of Related Art

Intraosseous infusion is a means for delivering fluids and drugs, most commonly through the tibia, to patients that must have emergency cannulization but lack peripheral vascular patency or, on occasion, are missing limbs due to accidents or other injuries. Intraosseous infusion may also be suitable where peripheral veins cannot be accessed for other reasons, such small size (as in children) or in low light conditions.

Although a number of products are sold or are in development for tibial intraosseous infusion, relatively little attention has been paid to sternal infusion, i.e., infusion performed on the manubrium bone of a patient. Sternal infusion may be particularly appropriate for military applications, such as in a battlefield environment, in which conditions are often difficult (as a result of ongoing combat, darkness, or other factors) or where there may be a large number of patients to be treated. The sternum is an attractive site for infusion or aspiration because it is a relatively soft bone, it has a wide marrow space of generally uniform thickness, it usually lies under only a thin layer of skin, and it is an accessible area in a patient lying in the prone position. In addition, it is more common in battlefield applications for peripheral venous access to be limited or unavailable due to loss of extremities.

One known product designed for sternal placement is called the F.A.S.T.® system, believed to be manufactured by Pyng Medical Corporation (Vancouver, B.C., Canada). This system, which appears to be described at least in part in U.S. Pat. No. 5,817,052, has a number of drawbacks, including relative bulkiness and a large number of components, as well as the fact that many of the components that could potentially be reusable are instead discarded after each use.

### SUMMARY OF INVENTION

Embodiments of the invention provide improvements to devices and methods for intraosseous infusion or aspiration, such as those described above.

A first aspect of the invention is directed to a device for infusion or aspiration that comprises a base and at least one needle positioned within the base, where the base includes one or more locators for positioning the infusion device in relation to one or more predetermined anatomical features.

In one embodiment of this aspect of the invention, an infusion or aspiration device comprises a driver that mates with or includes a base, where the base includes a feature for locating the base against a finger placed in the sternal notch of the patient. The base is also oblong, such that a long axis of the base may be aligned with the length of the sternum. By aligning the base with the sternal notch and with the axis of the sternum, the needle is automatically positioned above the desired insertion location.

A second aspect of the invention is directed to a device for infusion or aspiration that comprises a drive assembly and a plurality of needles positioned within the drive assembly.

In one embodiment of this second aspect of the invention, and infusion or aspiration device is constructed and arranged to allow for onboard storage of multiple infusion needles. The needles may be stored in a position to be rotated or translated within the device directly into a position suitable for insertion, or the needles may be withdrawn by the user from one portion of the device and inserted into another position suitable for insertion.

A third aspect of the invention is directed to a method for performing infusion or aspiration comprising the steps of establishing the location of the surface of a bone using the tip of a needle and then driving the needle into the bone a predetermined distance from the established location of the surface of the bone.

In one embodiment of this third aspect of the invention, a driver assembly and base are constructed and arranged to allow an intraosseous needle to be extended through the skin and soft tissue of a patient to a point at which the tip of the needle contacts the bone, establishing the location of the surface of the bone. The driver assembly is subsequently actuated, driving the needle a further fixed distance into the bone relative to the bone surface.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows a perspective view of one embodiment of an intraosseous infusion device according to the present invention;

FIG. 2 shows a cross section taken in the direction indicated by the line 2-2 of FIG. 1;

FIG. 3 shows an exploded perspective view of the infusion device of FIG. 1;

FIG. 4 shows an exploded perspective view the driver assembly of the infusion device of FIG. 1;

FIG. 5 shows a perspective view of the base assembly of the infusion device of FIG. 1;

FIG. 6 shows a perspective view of a cartridge assembly of the infusion device of FIG. 1; and

FIG. 7 shows a cross section taken in the direction indicated by the line 7-7 of FIG. 6.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” “having,” “containing,” “involving,” and variations thereof herein is meant to encompass the items listed thereafter and equivalents thereof, as well as additional items.

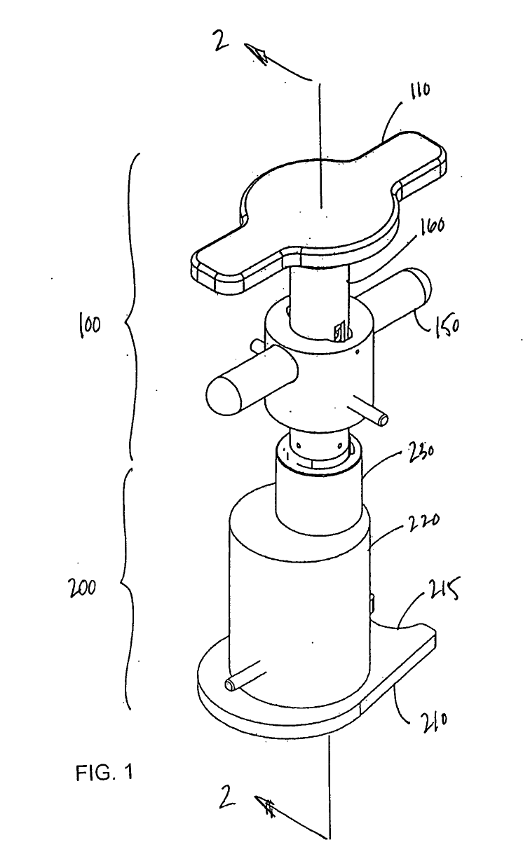

Referring now to the drawings, and more particularly to FIG. 1, there is generally indicated at 10 an infusion or aspiration device. For convenience, the inventive device may be referred to hereafter as only an “infusion device.” It should be understood, however, that such devices may be used for either infusion or aspiration.

The infusion device is designed to allow intraosseous infusion of fluids by facilitating the insertion of an intraosseous needle into bone. While the manubrium bone of the sternum is the preferred target for this particular device, it is understood that similar devices may be used for tibial or other placements, with appropriate changes in their configuration.

It is further contemplated that embodiments of the inventive device may be used for infusion or aspiration on non-human, i.e., animal, subjects.

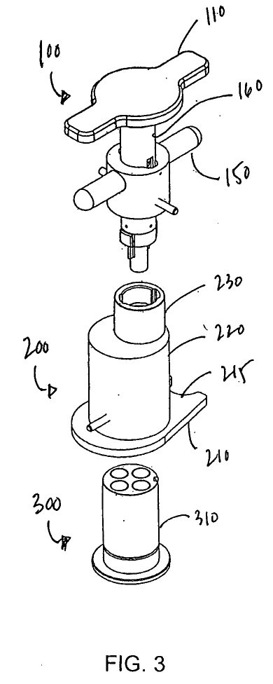

Referring to FIGS. 1 and 3, an exemplar infusion device 10 is composed of a driver mechanism 100, a base assembly 200, and a cartridge assembly 300.

The driver mechanism 100 is constructed to drive an intraosseous needle 400 though bone by applying force to the needle 400.

The inventive driver mechanism is not limited to the particular arrangement shown in the figures, nor is it limited to an arrangement in which the force is provided by a spring. Rather, the driver mechanism may take any appropriate form, be composed of any number of parts (or a single part), and the force applied to the spring may be applied by any appropriate source of energy including, by way of illustration only, an electric motor, compressed gasses or liquids, or by manual force, such as by the application of body weight or the thrust of a hand, arm, or other body part.

As will be discussed in more detail below, in one particular embodiment, the driver mechanism 100 is adapted to allow the needle 400 to be inserted into the soft tissue of a subject to the point at which the needle 400 contacts the surface of the bone at or about the desired insertion site. The driver mechanism 100 is further configured to subsequently drive the needle a predetermined further distance into the bone. The predetermined further distance may be permanently fixed in the construction of the device or, in some cases, may be adjustable by the user. In some embodiments, this process may be facilitated by the use of an appropriately configured base assembly 200, as described below.

In one alternative embodiment, the driver mechanism is simply a T-shaped handle that is adapted to be held in the hand of a user. The force is applied to the needle by way of the user pressing the device in the desired direction.

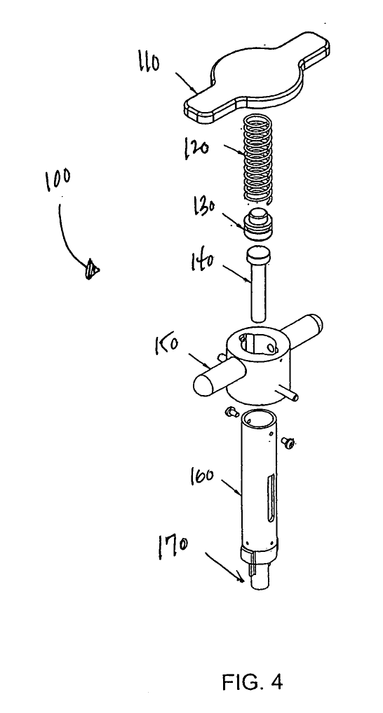

Referring to FIG. 4, the exemplar driver mechanism 100 of FIG. 1 is composed of a palm support 110, a spring 120, a hammer 130, a driver tip 140, a trigger assembly 150, a driver sheath 160, and a driver base 170. The palm support 110 is mounted on an upper end of the driver sheath 110, the spring 120, hammer 130, and driver tip 140 are positioned one above the other within the driver sheath, the trigger assembly 150 surrounds the driver sheath 160, and the driver base 170 is affixed to a bottom of end of the driver sheath 110.

In operation, pressure is applied to the driver mechanism 100 to move the needle 400 through the skin to contact the bone at the desired insertion site. The interface between the driver mechanism 100 and the base assembly 200 of this embodiment allows the driver mechanism 100 to move into and out of the base assembly 200 when in one orientation, while a second orientation locks the driver in its location with respect to the base assembly 200. This combination of motion and locking may be achieved in any of several ways, including by segmented teeth, a cam lock, a compression fitting, or an eccentric cross section. In the device shown in FIG. 1, the relative motion and locking is achieved by a segmented threaded fitting.

When the interface is in the movable orientation, driver mechanism 100 may be moved such that the needle 400 passes through the skin and soft tissue until the needle tip contacts the bone. Once the needle 400 contacts the bone, the driver mechanism 100 may be locked with respect to the base using one of the methods discussed above. Locking the driver mechanism 100 fixes the needle position and establishes the reference point (i.e., the location of the surface of the bone) for firing the needle an additional predetermined distance into the bone.

The user's palm is then placed against the palm support 110 and the user's fingers are used to draw the trigger assembly 150 in the direction of the palm support 110. Trigger assembly 150 engages hammer 130 by means of a catch spring, such that hammer 150 is drawn upwards with trigger assembly 150, in the direction of the palm support 110, compressing spring 120.

When the trigger assembly 150 reaches a predetermined position, a ramp contacts the catch spring and causes the trigger assembly 150 to be disengaged from the hammer 130. With the hammer 130 disengaged from the trigger assembly 150, the spring 120 is no longer constrained and drives the hammer 130 downward. In turn, the hammer 130 strikes the driver tip 140 and drives it downwards until its travel is arrested by an upper surface 175 of the driver base. As will be described in more detail below, the driver tip 140 drives the needle 400 into the bone.

When the trigger assembly 150 is returned to the starting position, it reengages with the hammer 130 in preparation for the next needle placement.

In some embodiments, the driver tip 140 may be configured such that it maintains constant contact with the needle 400 during the actuation of the device, so as to avoid severe impacts to the needle that could cause damage to the bone.

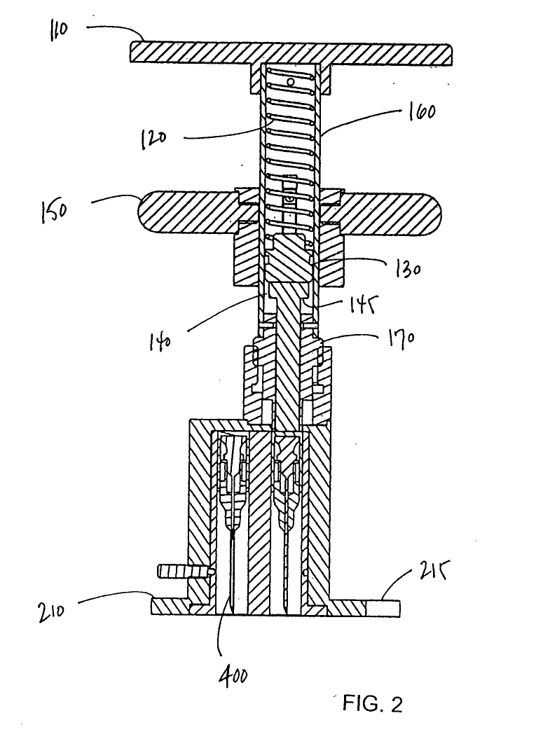

As described above, the driver tip 140 may also serve the function of controlling the insertion depth of the needle 400. The distance the driver tip can travel is defined by the gap between the head 145 of the driver tip 140 and the driver base 170, as can be seen in FIG. 2. This gap can be designed according to the relevant anatomical and physiological data for the bone that is to be targeted. In one preferred embodiment, the gap is on the order of 5 mm, so as to be appropriate for the use with the human sternum. The device can also be designed such that the gap is adjustable to accommodate different targeted bones or unusual anatomies.

In the illustrated embodiment, the driver tip 140 is constructed of stainless steel, although it is contemplated that other metals may also be used, as may other materials, such as certain plastics, ceramics, or composites. Considerations in selecting an appropriate material include strength, weight, and corrosion resistance. In other embodiments, the drive tip 140, if employed, may have different shapes or sizes.

The spring 120 of the illustrative embodiment is a conventional metal compression spring, although other types of springs (and other energy sources) may be used in other embodiments. One important aspect of the spring or other energy source is the force delivered upon actuation of the device. If the force provided is too low, the needle will not penetrate the bone or will not penetrate to the desired depth. On the other hand, if the force delivered is too high, the needle may actually crack the bone upon entry or be driven past the desired insertion depth. In addition, a spring capable of delivering a higher force may be more difficult for a user to compress.

The level of force applied, and the source, may vary depending upon the application. In general, it is desirable to use the lowest level of force that will reliably penetrate the bone to the desired depth for a given site. It has been found in connection with the illustrated embodiment that, based on testing in animal bone and with synthetic human tissue, an appropriate force for use on the human sternum and with a conventional intraosseous needle may be provided by a 2.5 inch long compression spring with a spring constant of approximately 9.5 lbs/in. It should be appreciated, however, that the appropriate amount of force will vary depending upon the particular application and with factors such as the desired application site and the size of the needle.

The palm support 110, hammer 130, trigger assembly 150, and a driver sheath 160 may be made from a number of appropriate plastic or metal materials. Each of these elements may take on any suitable shape, with important design considerations including size, as the overall device should be as compact and as is feasible, and ergonomics, as the device should be as comfortable and easy to use as possible. In some cases, various parts of the driver mechanism 100 may be provided with a coating or covering to provide comfort to the user or the patient, or to protect or reduce friction in the device.

In the illustrative embodiment of FIG. 1, the base assembly 200 is configured to receive the driver mechanism 100, in particular the driver base 170, and to facilitate its proper positioning for insertion of the needle 400.

While not all embodiments of the invention include a base assembly 200, a base may provide advantages in certain applications. In particular, the base assembly may include a lower surface or other feature adapted to contact the skin of the patient and provide stability. A base assembly may also aid in the positioning of the driver mechanism relative to a given anatomical feature, in positioning the driver mechanism perpendicular or approximately perpendicular to the surface of the bone at the desired insertion site, and/or in the positioning the needle 400 relative to the surface of the bone. In some embodiments, the base assembly may also provide for storage of extra needles 400.

As described above, the base assembly 200 may be constructed and arranged to allow the driver mechanism 100 to move into and out of the base assembly 200 when in one orientation, while a second orientation locks the driver in its location with respect to the base assembly 200.

Like the driver mechanism, the base assembly may take any appropriate shape and may be composed of one or more parts formed separately or integrally. As noted, a base assembly is not a necessary element of the inventive device, as certain embodiments may function properly and intentionally without a base assembly. In some embodiments, the driver mechanism may be configured to be used either with or without a base assembly.

In still further embodiments, the base assembly may be formed as part of the driver assembly, either integrally or as separate parts temporarily or permanently attached by integral threads, threaded or non-threaded fasteners, adhesives, or other appropriate means.

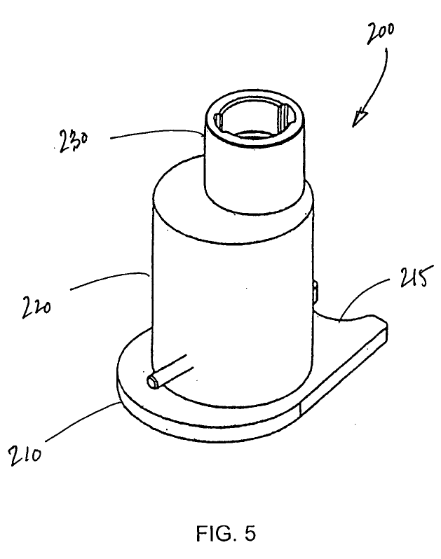

As seen in FIG. 5, the base assembly 200 of the exemplar embodiment comprises a foot portion 210, a body 220, and a lock ring 230.

The lock ring 230 is adapted to receive the driver base 170, as can be appreciated from FIG. 3. In this embodiment, the lock ring 230 is formed of a separate part from the body 220, but it should be understood that the lock ring 230, if employed, may also be integral to the body 220 and may assume other configurations. In this particular embodiment, the lock ring 230 includes a threaded opening in the top that receives the threaded end of the driver base 170, although such an arrangement is also not a necessary element of the invention.

The body 220 of this embodiment is cylindrical in shape and receives the lock ring 230 in its top face. As described more fully below, this body 200 also includes a hollow interior adapted to receive a cartridge assembly 300 that carries one or more intraosseous needles 400. It should again be appreciated that this embodiment is illustrative only and that the body 200 may in other embodiments be constructed in various other configurations or, in some embodiments, may be omitted entirely.

The foot 210 of this embodiment is formed integrally with the body 220, is oblong, is longer and wider than the body portion, and has a relatively low profile. The length and width of the foot 210 provide stability and aid in maintaining the driver portion in a roughly perpendicular orientation to the face of the bone at the insertion site. As with body 200 and the lock ring 230, however, the foot 210 may take on different shapes in different embodiments and may, in some cases, be omitted entirely.

In some embodiments, the infusion device 10 may include one or more locators for positioning the drive mechanism 100 and/or needle 400 with respect to one or more particular anatomical features. A non-exhaustive list of possible locators include the size or shape of all or a particular portion of the infusion device 10, bumps, knobs, or other protrusions, notches, recesses, holes, windows, and/or any other construction or feature that allows the device to be positioned relative to a given anatomical feature. By anatomical feature, it is meant a physical feature of a human or animal that generally exists across the species or a subset of the species, a particular example of which is the sternal or tracheal notch on a human being. Another example of an anatomical feature is the centerline of the sternum. The surface of the skin itself is not considered to be an anatomical feature.

In the embodiment shown in FIG. 1, one locator is a notch 215 formed as part of the foot 210. In operation, the user of the device places a finger into the sternal notch of the patient and then slips the notch 215 around the finger, with the foot 210 placed against the chest of the subject. A second locator is the oblong shape of the foot 210, where the long axis of the foot 210 can be aligned with the length of the sternum (or the body as a whole). As a result of aligning the notch 215 with the finger in the sternal notch and the long axis of the foot 210 with the length of the sternum, the needle is automatically positioned above a predetermined insertion location in the manubrium bone. In one embodiment, the fixed distance between the sternal notch and the needle position is approximately 2 cm. This fixed distance may vary in other embodiments and, in some cases, the distance could be adjustable.

This particular pair of locators has the advantage of being easy to use even under difficult conditions, such as where there is little light. It also has the advantage of providing a relatively high degree of accuracy, as the first locator fixes the device relative to a predetermined point and the second locator fixes the device relative to a predetermined angle. The position of the needle is subject to multiple constraints represented by the surface of skin, the sternal notch, and the axis of the sternum (or body). Accuracy is higher than a case in which only a single locator (e.g., the sternal notch) is used.

In other embodiments, locators could include a ring thorough which a finger could be inserted to engage the sternal notch or, in another embodiment, a rounded or other protrusion on the lower surface of the base that directly engages the sternal notch. Other locators would be appropriate as well, provided that they allow the device to be positioned relative to at least one predetermined anatomical feature.

In still further embodiments, all or some portion of the locator could be formed as part of the driver assembly 100. For example, the palm support 110 or trigger assembly 150 could be constructed and arranged to assist with alignment of the device with the length of the sternum.

All or part of the base assembly may be made from a number of appropriate plastic, metal, or composite materials. In some cases, parts of the base assembly may be provided with a coating or covering to provide comfort to the patient. The base plate may also include illuminated indicia to facilitate placement and use in low light conditions.

In addition, the base assembly may contain one or more adhesive-coated layers that adhere to the skin and act as a bandage material. Such layers could act as an anchorage for the needle during and following insertion. The base assembly may in some cases be configured to accommodate the size and/or age of the individual.

In the illustrative embodiment of FIG. 1, the base assembly 200 is configured to receive a cartridge assembly 300 that includes a plurality of intraosseous needles.

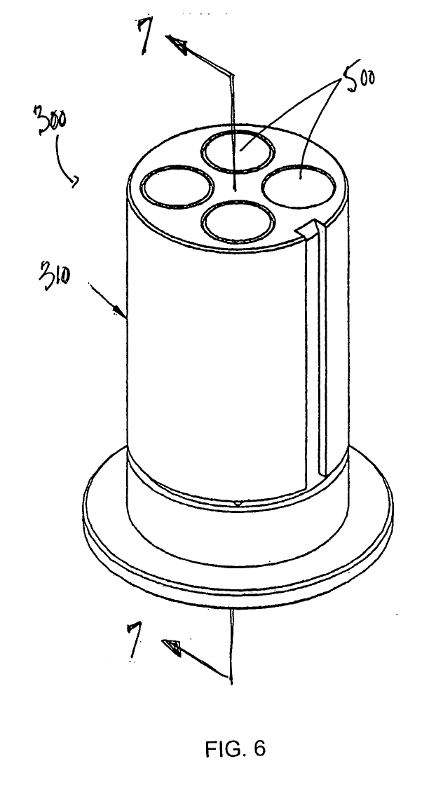

A cartridge assembly 300 such as that shown in FIG. 6 may be a disposable item that carries a certain number of needles or it may be reusable and adapted to receive individual needle packages 500. An advantage of a disposable cartridge assembly 300 is that it avoids the difficulties associated with handling individual needle packages 500, which can be difficult under certain conditions.

Like the other elements on the device, the cartridge assembly 300 may take any appropriate shape and may be composed of one or more parts. In addition, it should be understood that a cartridge assembly 300 is not a necessary element, in particular because not all embodiments of the invention even require multiple needles.

As seen in FIG. 6, the cartridge assembly 300 of the exemplar embodiment comprises a cartridge body 310 adapted to house multiple needle packages 500 positioned in wells within the cartridge body 310. The cartridge body 310 itself fits into the hollow interior of the body 220 and may be retained there by friction or other appropriate means.

In the illustrated embodiment, the cylindrical shape of the cartridge body 310 allows it to be rotated to bring the individual needles into position to be driven by the driver mechanism 100. Specifically, the axis of the driver mechanism and, in particular, the driver tip 140, is offset from the axis of the cartridge body 310, such that rotation of the cartridge body 310 will bring the individual needle packages 500 into axial alignment with the driver tip 140. In this position, the driver tip 140 may be advanced to move an individual needle 400 first to a position in contact with the surface of the bone and, then, to drive the needle 400 a predetermined further distance into the bone, as described above.

After a needle is driven into the bone, the infusion device may be lifted from the patient and the cartridge assembly 300 rotated, bringing a fresh needle package 500 into alignment with the driver tip 140. Once the driver mechanism 100 is reset, as also described above, the device is ready to be used again. In some embodiments, the cartridge body 310 may rotate automatically after each use and/or may lock to prevent the attempted firing of more needles than are contained in a single cartridge body 310.

Again, however, it should be appreciated the cartridge assembly 300 of the illustrated embodiment is just one aspect of one embodiment of the invention and that the invention contemplates needle cartridges of different shapes, sizes, and modes of operation, arrangements for the storage of multiple needles onboard that do not include any type of cartridge, and arrangements in which only one needle is stored onboard.

In another embodiment, for example, needle packages 500 could be arranged in a linear fashion, either side-by-side, so as to be able to be translated within the device directly into a position suitable for insertion, or stacked end-to-end. In some embodiments, the additional needles 400 or needle packages 500 might be placed so as to be withdrawn by the user from one portion of the device and inserted into another position suitable for insertion.

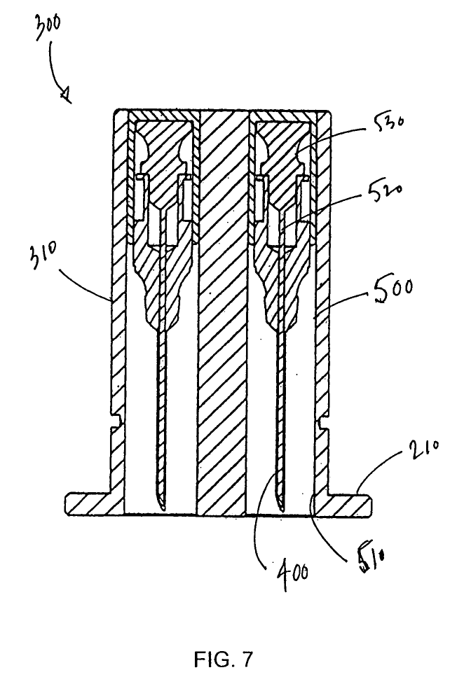

As described above, the cartridge assembly 300 of exemplar device of FIG. 1 includes a plurality of needle packages 500. As can be seen in FIG. 7, each needle package 500 of this embodiment includes a sterile casing 510, a needle 400, and a rigidifying stylet 520 passing down the hollow shaft of the needle 400. The needle assemblies 500 of the illustrative embodiment, which in this case are cylindrical but need not be, may provide a guide to the needle 400 as it passes out of the infusion device 10 and into the target.

The needle 400 is contained within a sterile casing 510 that is configured to be pierced by the needle 400 upon application of force by the driver tip 140. The side wall of the sterile casing 510 may be composed of a material such as polypropylene, polystyrene, polycarbonate, or other material that may be hermetically sealed with a packaging film on each end to guarantee sterility.

The stylet 520 of the illustrative embodiment is fixed to a cap 530 positioned at the distal end of the sterile casing 510. In operation, the driver tip 140 presses through the upper surface of the sterile casing 510 and drives the stylet 520 and the needle 400 into the bone. The stylet 520 is removed after placement of the needle and prior to attachment of fluid lines to the patient. The cap 530 may be attached to the needle by means of a standard luer fitting, although bayonet, threaded, press-fit, or other methods may be used.

In one particular embodiment, the needle 400 comprises a 15 to 19 gauge stainless steel needle with a bone piercing tip at the proximal end. The tip may be beveled to facilitate insertion into the bone.

In some embodiments, the needle 400 may have bone-cutting threads to facilitate its movement through the bone. In these or other embodiments, the needle 400 may have a “keyed” shape that allows the needle 400 to be anchored in the bone through a small rotation of shaft. In some embodiments, materials other than stainless steel, such as shape memory alloys (e.g., Nitinol), may be used to provide the needle with various properties, such as elasticity to facilitate a low profile after insertion. The use of a shape memory alloy could allow the needle 400 to bend slightly upon removal of the stylet 520. In some embodiments, it may also be useful to employ bioresorbable polymers, such as polylactide/glycolide copolymers, polyorthoesters, or other polymers that would allow the needle tip or body to remain in the patient after use until reabsorbed. Components of the needle may also be composed of a hardened bone material such as a polymer-coated or metal-coated hydroxyapatite that is reabsorbed over time after placement.

In some embodiments, the needle assemblies 500 may include an antiseptic or other material within the package. In such embodiments, antiseptic or other material coats the surface of needle 400 as the needle is driven from the needle assembly 500. The material may also be carried out of the package and onto the target surface where it may counteract unclean conditions. Appropriate materials may include an ointment base or other commercialized base containing broad-spectrum antibiotics such as polymyxin, erythromycin, or neomycin. In other embodiments, it is anticipated that other agents may be delivered in this manner, such as local anesthetics, wound-healing agents, or even sealants for sealing the puncture made by the needle as it enters the soft tissue.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A device for infusion or aspiration comprising:
a base; and
at least one needle positioned within the base;
wherein the base comprises a locator for positioning the device in relation to a predetermined anatomical feature.

2. The device of claim 1, wherein the predetermined anatomical feature is the sternal notch.

3. The device of claim 2, wherein the base comprises a second locator for positioning the infusion device in relation to a second predetermined anatomical feature.

4. The device of claim 3, wherein the second predetermined anatomical feature is the centerline of the sternum.

5. The device of claim 1, further comprising:
a driver constructed and arranged to drive the needle into bone.

6. The device of claim 5, wherein the base is integral to the driver.

7. The device of claim 5, wherein the driver is moveable with respect to the base.

8. The device of claim 5, wherein the driver is spring-actuated.

9. The device of claim 1, wherein the needle is an intraosseous infusion needle.

10. The device of claim 1, wherein a plurality of needles are positioned within the base.

11. A device for infusion or aspiration comprising:
a drive assembly; and
a plurality of needles positioned within the drive assembly.

12. The device of claim 11, further comprising a locator for positioning the device in relation to a predetermined anatomical feature.

13. The device of claim 11, wherein the plurality of needles are positioned for sequential use without removal from the device.

14. The device of claim 11, wherein the drive assembly is spring-actuated.

15. A method for performing infusion or aspiration comprising the steps of:
establishing the location of the surface of a bone using the tip of a needle; and
driving the needle into the bone a predetermined distance from the established location of the surface of the bone.

16. The method of claim 15, further comprising the step of establishing the location for insertion of the needle into the bone in relation to a predetermined anatomical feature.

17. The method of claim 16, further comprising the step of establishing the location for insertion of the needle into the bone in relation to a second predetermined anatomical feature.

18. The method of claim 15, wherein the step of driving the needle into the bone is performed by a spring-actuated driver.

19. The method of claim 15, further comprising the step of using the needle for intraosseous infusion.

20. A device for infusion or aspiration comprising:
a first locator for positioning the device in relation to a first predetermined anatomical feature and;
a second locator for positioning the device in relation to a second predetermined anatomical feature.

21. The device of claim 20, wherein the first locator fixes the device relative to a predetermined point and the second locator fixes the device in a particular angular orientation.
